(19) United States
(12) Plant Patent Application Publication
(10) Pub. No.: US 2002/0152508 P1
(43) Pub. Date: Oct. 17, 2002

(54) Red miniature rose plant named 'Jentara'

(76) Inventor: Svend Jensen, Bjert (DK)

Correspondence Address: CAL-EUROPE, Att: PER HANSEN, 905 SOUTH PATTERSON AVENUE, P.O. BOX 61524, SANTA BARBARA, CA, 93160, US

(21) Appl. No.: 09/772,695

(22) Filed: Jan. 31, 2001

(51) Int. Cl. A01H 5/00

(52) U.S. Cl. PLT/117

(57) ABSTRACT

A new white miniature rose plant which has abundant non-fading flowers and good keepability. The variety successfully propagates from softwood cuttings and is suitable for year round production in commercial glasshouses. The new and distinct variety has shown to be uniform and stable in the resulting generations from such asexual propagation.

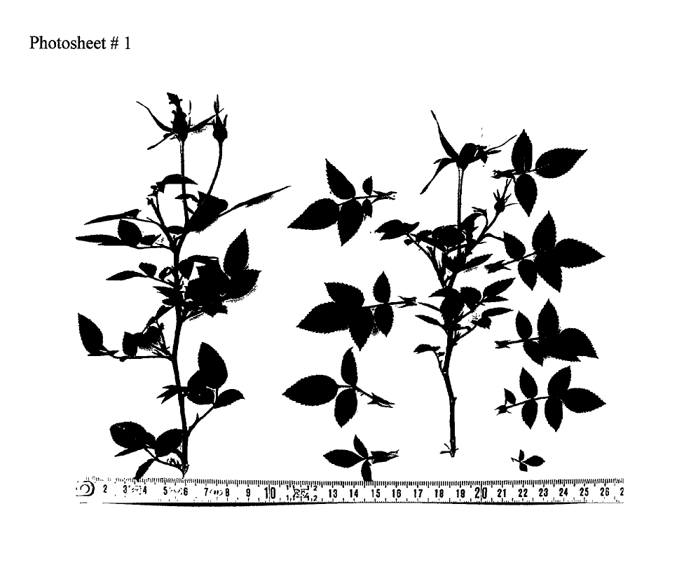

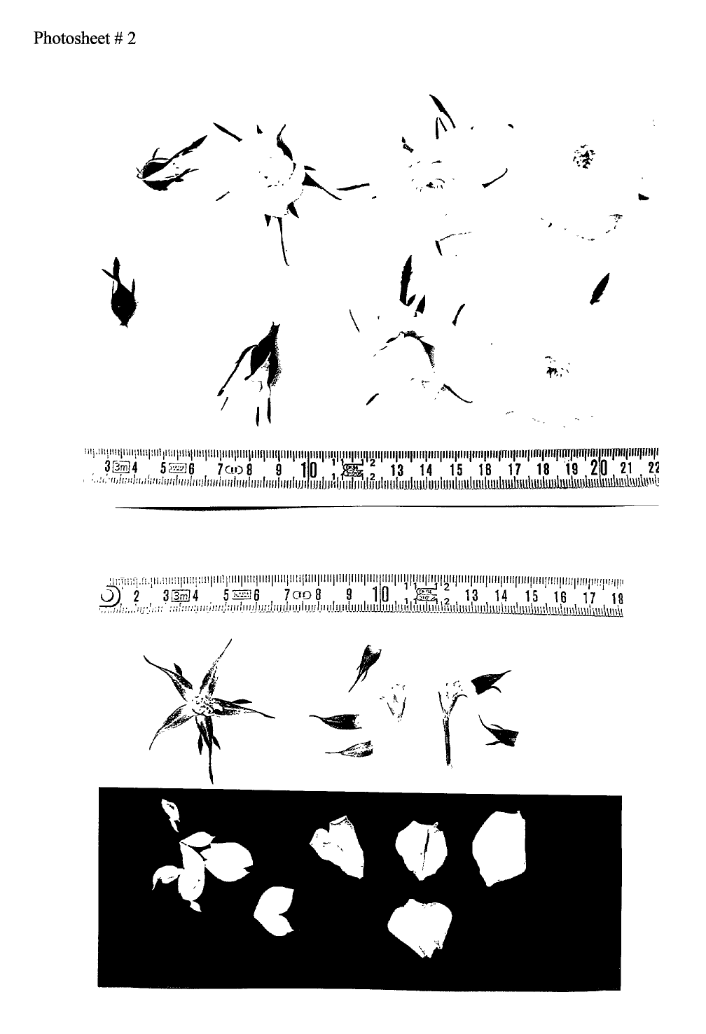

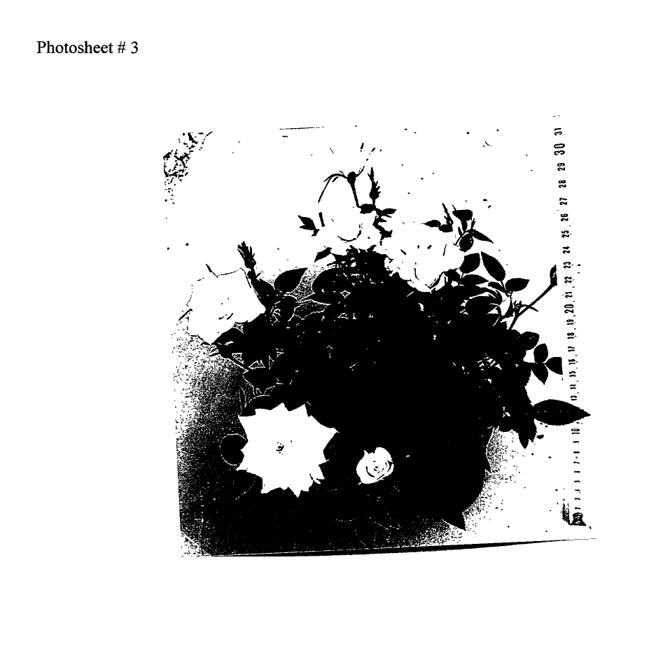

### BACKGROUND OF THE INVENTION

The present invention constitutes a new and distinct variety of miniature rose plant which was developed by artificially pollinating an unnamed seedling with an unnamed seedling.

The two parents were crossed in the summer of 1996 and the resulting seed was sown in autum, 1996 in a controlled glasshouse environment. The new variety is named ‘Jentara’ The objective of the hybridization of this rose variety for commercial greenhouse culture was to create a new and distinct variety with:

1. Uniform and abundant flowers with excellent keepability;

2. Attractive long lasting foliage and compact growth,

3. Year round flowering under glasshouse conditions;

4. Suitability for production from softwood cuttings in pots; and

5. Durable flowers and foliage which make the variety suitable for distribution in the floral industry.

This combination of qualities was not present in previously available commercial cultivars of this type and distinguish ‘Jeantara’ from all other varieties of which we are aware.

The seeds from hybridization were planted in a controlled environment and evaluations were conducted on the resulting plants. ‘Jentara’ was selected by Svend Jensen in He's development program in Bjert, Denmark, in june 1997..

### SUMMARY OF THE INVENTION

‘Jentara’ is tested at DANROSE nursery,Dk-6051 Bjert, Denmark in 1998, and at Por La Mar Nursery, 600 S. Patterson, Santa Barbara, Calif. 93160, USA in 1999. Have here proven to be stabile by propagation with cuttings in several generations. ‘Jentara’ is a low and compact miniature rose with medium vigor. The shelflife has been tested to be good.

### BRIEF DESCRIPTIONS OF THE DRAWINGS

The accompanying color illustrations show as true as is reasonably to obtain in color photographs of this type, the typical characteristics of the buds, flowers, leaves, stems, and a plant of ‘Jentara’. Specifically illustrated in sheet 1:

1. Flowering stem w. leaves

2. Leaves and stem detached.

Sheet 2:

3. Flower bud, partially opened bud, and to open bloom;

4. Sepals, pistils & stamens;

5. Petals detached;

Sheet 3:

6. Specifically illustrated an entire blooming plant in a 10.5 cm pot.

### DETAILED DESCRIPTION OF THE VARIETY

The following is a detailed description of ‘Jentara’, as observed in its growth in glasshouses inBjert, Denmark, and Glass-/polyhouses in Santa Barbara Calif. The cultivation at both location was conducted with 20 to 25 dgC Temperature, and 250 Klux light sum per day, and minimum 4 hours night. Descriptions were made from plants treated with growth regulators normally used in the greenhouse production process. The growth regulator Paclobutrazol was applied at 15-30 ppm weekly beginning at a plant age of 9 weeks. The peduncle lengths mentioned may actually be sorter and the foliage color several shades darker than on untreated specimens. Color references are made using The Royal Horticultural Society (London, England) Colour Chart.

Classification:

Botanical: Rosa hybrida.

Commercial: Miniature.

| | |
|---|---|
| Characteristic | Application variety |
| Crop time | 12 weeks |
| Appropriate container used | 10 cm |
| Plant shape | Rounded |
| Growth habit | Upright |
| Plant height | 28 cm |
| Plant diameter or area of spread | 20 cm |
| Growth rate/vigor | Rapid/moderate |
| Branching habit | Very freely |
| Pinching required? | Yes |
| Numbers of lateral branches | 3 to 4 |
| Lateral branch length | 16.5 cm |
| Lateral branch diameter | 2 mm |
| Internodie length | 10-20 mm |
| Stem color | RHS, Green group, NO.: 137A |
| Stem pubescence | None |
| Other Plant/stem characteristics | None |
| Leaf arrangement | Alternate |
| Compound or single? | Compound |
| Quantity of leaves per lateral branch | 6 to 7 |
| Quantity of leaflets per leaf | 3 to 5 |
| Leaf(leaflet) shape | Ovate |
| Leaf (leaflet) Tip | Accuminate |
| Leaf (leaflet) base | Obtuse |
| Leaf/leaflet length | 50 to 75 mm/20 to 30 mm |
| Leaf/leaflet width | 38 to 45 mm/8 to 20 mm |
| Leaf (leaflet) texture | Smooth |
| Leaf pubescence? Which side? | None |
| Leaf(leaflet) margin. | Serrate |
| Venation pattern | Pinnate |
| Leaf color, young, upper side | RHS, Yellow-Green group, No.: |
| | 146B with reddish edge. |
| Leaf color, young, under side | RHS, Yellow-Green group, No.: |
| | 146B, with reddish edge. |
| Leaf color, mature, upper side | RHS, Green group, No.: 137A.. |
| Leaf color, mature, under side | RHS, Green group, No.: 138B. |
| Vein color, upper side | RHS, Green group, No.: 137A. |
| Vein color, under side | RHS, Green group, No.: 137D. |
| Petiole length | 15 to 25 mm |
| Petiole diameter | 1 mm |
| Petiole color | Upper side: RHS, Green group, |
| | No.: 137A, W. reddish tone |
| | Under side: RHS, Green group, |
| | No.: 137A, W. reddish tone |
| Durability of foliage to stresses | Stiff |
| Stipules | Yes. 6 mm, RHS, Yellow-Green |
| | group, No.: 147A, w. reddish tone |
| Thorns | None |
| Other foliage characteristics | None |
| Flower arrangement | Single to corymb |
| Flower form | Filled |
| Flowering habit | Recurrent |
| Quantity of flowers per lateral stem | 1 to 3 |
| Quantity of flowers and buds | 12 to 14 |
| per plant | |
| Natural flowering season | Year round |
| Time to flower or response time | 35 days as avr. For the year. |
| Rate of flower opening | Moderate |
| Frangrance | None |
| Flower aspect | Upon opening ovoid. Complete |
| | open: flat to rounded. Petals |
| | bending backward |
| Flower shape | Flat to rounded |
| Flower diameter | Up to 40 to 50 mm |
| Flower depth | Up to 15-20 mm |
| Flower longevity on plant | 18 days |
| Flower longevity as a cut flower | Unknown |
| Persistent or self cleaning? | Persist on the plant |
| Petal appearance | Dull to bright |
| Petal texture | Smooth |
| Petal arrangement | 5 per circle |
| Petal number | 75 |
| Petal fused? | No |
| Petal shape | Orbicular |
| Petal margin | Entire |
| Petal tip | Cuspidate |
| Petal length | 12 to 28 mm |
| Petal width | 8 to 25 mm |
| Petal color, when opening, upper side | RHS, White group, No.: 155C, |
| | w. green center strip |
| Petal color, when opening, under side | RHS, White group, No.: 155C, |
| | w. green center strip |
| Petal color, fully opened | RHS, White group, No.: 155C, |
| | w. green center strip |
| Petal color, fading to | RHS, White group, No.: 155C, |
| | w. green center strip |
| Petaloids? | Yes |
| Petaloid appearance | Dull |
| Petaloid arrangement | Spread |
| number of petaloids | 5 |
| Petaloid shape | Linear |
| Petaloid margin | Entire |
| Petaloid length | 4 to 5 mm |
| Petaloid width | 2 to 3 mm |
| Petaloid color, immature | RHS, White group, No.: 155C. |
| Petaloid color, mature | RHS, White group, No.: 155C. |
| Sepal appearance | Semi glossy |
| Sepal texture | Stiff and hairy |
| Sepal arrangement | one circle w. 5 |
| Number of sepals | 5 |
| Sepal shape | Linear |
| Sepal margin | Entire with a few leaflets appendix |
| Sepal Tip | Acuminate |
| Sepal color, upper side | RHS, Green group, No.: 138A |
| Sepal color, under side | RHS, Green group, No.: 137C |
| Calyx shape | Funnel shaped |
| Calyx length | 6-7 mm |
| Calyx diameter | 5-6 mm |
| Peduncle length | Apr. 50 mm |
| Peduncle angle | Straight |
| Peduncle strength | Stiff |
| Peduncle color | RHS, Green group, No.: 137C, |
| | w. tiny reddish prickles. |
| Flower bud length | 20-25 mm |
| Flower bud diameter | 12-15 mm |
| Flower bud shape | Urceolate |
| Rate of opening | Medium |
| Flower bud color | RHS, White group, No.: 155C |
| Stamen number | 10 |
| Anther shape | Elliptical to ovate |
| Anther size | 2 to 3 mm |
| Anther color | RHS, Yellow group, No.: 13B. |
| Amount of pollen | Little |
| Pollen color | RHS, Yellow group, No.: 13B |
| Pistil number | 30 |
| Pistil length | 4-5 mm |
| Stigma shape | Rounded |
| Stigma color | RHS, White group, No.: 155C. |
| Style length | 3-4 mm |
| Style color | RHS, White group, No.: 155C, |
| | w. heavy tone of Red-purple |
| | group, No.: 67A |
| Ovary color | RHS, White group, No.: 155C. |
| Other flower characteristics | None |
| Seed development? | Unknown |
| Seed length | Unknown |
| Seed diameter | Unknown |
| Seed color | Unknown |
| Disease resistance | Unknown |
| Disease susceptibility | Susceptible to Powdery mildew |

1. A new and distinct variety of rose plant of the miniature class, substantially as herein illustrated and described, as a distinct and novel rose variety due to its abundant flowers with excellent keepability, attractive long lasting foliage and compact growth, year round flowering under glasshouse conditions, suitability for production from softwood cuttings in pots, and durable flowers and foliage which make the variety suitable for distribution in the floral industry.
